(19) United States
(12) Patent Application Publication
(10) Pub. No.: US 2011/0286942 A1
(43) Pub. Date: Nov. 24, 2011

(54) Topical Composition Comprising a Chaotrope

(75) Inventors: Anjing Lou, Seymour, CT (US); Qiang Qiu, Trumbull, CT (US)

(73) Assignee: CONOPCO, INC., D/B/A UNILEVER, Englewood Cliffs, NJ (US)

(21) Appl. No.: 12/784,046

(22) Filed: May 20, 2010

(51) Int. Cl. A61K 8/41; A61Q 17/04; A61Q 15/00; A61K 31/13

(52) U.S. Cl. 424/59; 424/65; 514/674

(57) ABSTRACT

Topical compositions with a chaotrope are described. The compositions can comprise guanidinium chloride or a derivative thereof and an active, and they unexpectedly display rapid active delivery and improved active functionality upon application.

### FIELD OF THE INVENTION

The present invention is directed to a topical composition comprising a chaotrope. More particularly, the invention is directed to a topical composition comprising a chaotrope and an active whereby the chaotrope does not irritate the skin of the consumer and does, surprisingly, result in better transdermal delivery of the active as well as enhanced active functionality.

### BACKGROUND OF THE INVENTION

One of the main disadvantages in topical active delivery is low and/or slow penetration of active via transdermal delivery. Poor penetration of active invariably means poor active functionality. Several techniques have been investigated to increase active penetration rates across skin. Pharmaceutical researchers, for example, have sought effective means of introducing drugs into the bloodstream by applying them to skin with a penetration enhancer. Such penetration enhancers include agents like dodecyl pyrrolidone, dimethyl lauramide, dimethyl sulfoxide as well as dodecylazacycloheptan-2-one.

Methods that use electrical current have been employed in order to enhance the penetration of molecules into the skin. These methods include iontophoresis, a technique where charged molecules are driven into the skin by a small direct current. Other electrical methods for penetration enhancement include phonophoresis (ultrasound energy), electroporation (application of short electrical pulses) and use of photomechanical waves (laser-generated stress). Synergy between chemical enhancers and electrically assisted methods has also been tried as have methods that bypass the stratum corneum with microneedles.

Notwithstanding the existing measures taken to deliver actives through the skin, there is a need for an ideal means to enhance transdermal delivery of actives in a manner which is safe, affordable, convenient and environmentally friendly. This invention, therefore, is directed to a topical composition that allows for quick delivery of actives. More particularly, the invention is directed to a topical composition comprising a chaotrope and an active whereby the chaotrope surprisingly results in better transdermal delivery of the active while at the same time enhancing the efficacy of the active. Use of the chaotrope is not invasive, does not irritate the skin and does not result in symptoms that would be considered unpleasant to the ordinary consumer.

### ADDITIONAL INFORMATION

Efforts have been disclosed for improving transdermal delivery of agents. In U.S. Pat. Nos. 4,405,616, 4,886,783 and 5,118,845, penetration enhancers suitable for use in transdermal delivery systems are described.

Still other efforts have been disclosed for demonstrating deposition of drugs into and across skin. In U.S. Pat. No. 5,720,948, non-ionic surfactant emulsion vehicles are described for their use in drug deposition through skin.

Even other efforts have been disclosed for transdermal drug delivery. In Current Drug Delivery, 2005, 2, pp. 23-33, penetration enhancement techniques are described.

None of the additional information above describes a topical composition comprising a chaotrope for improving the transdermal delivery of an active in a topical composition.

### SUMMARY OF THE INVENTION

In a first aspect, the present invention is directed to chaotrope and active additive comprising:

(a) a chaotrope represented as:

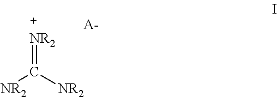

and optionally a chaotrope represented as:

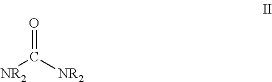

where each R is independently H, C₁₋₁₀ alkyl, or aryl, and

A⁻ is a halide; and

(b) an active.

In a second aspect, the present invention is directed to an end use composition comprising the additive of the first aspect of the invention.

In a third aspect, the present invention is directed to a method for improving a skin characteristic with the end use composition of the second aspect of this invention.

All other aspects of the present invention will more readily become apparent upon considering the detailed description and examples which follow.

Active, as used herein, is meant to include agents suitable to enhance a skin characteristic. Active, for example, includes but is not limited to a skin lightening, self-tanning, moisturizing, deodorizing, antiperspirant, anti-acne, wrinkle reducing agent or mixture thereof. In a preferred embodiment, however, the active used in this invention is a self-tanning agent.

Skin, as used herein, is meant to include skin on the face, neck, chest, back, arms, hands, legs, buttocks and scalp. End use composition is meant to mean a composition ready for a consumer to topically apply so that a skin characteristic of choice may be enhanced (i.e., improved). In a preferred embodiment, the end use composition comprises a high internal phase emulsion (HIPE) comprising at least about 65% by weight water based on total weight of the composition. Alkyl, as used herein, is meant to include linear, cyclic and/or branched alkyl groups.

Comprising, as used herein, is meant to include consisting essentially of and consisting of. All ranges identified herein are meant to include all ranges subsumed therein if reference to the same is not explicitly made.

### DETAILED DESCRIPTION OF THE PREFERRED EMBODIMENT

The only limitation with respect to the chaotrope that may be used in this invention is that the same is suitable to be formulated in a composition that may be topically applied to the skin of humans.

Preferred chaotropes suitable for use are typically represented by the formula:

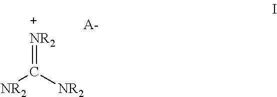

where each R is independently H, C₁₋₁₀ alkyl, or aryl, and

A⁻ is a halide.

Optional chaotropes suitable for use with the choatropes represented by formula I include those represented by the formula:

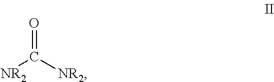

where R is as previously defined.

In an often preferred embodiment, each R is hydrogen and A⁻ is a chloride anion and the chaotrope employed in this invention is carbamimidoylazanium chloride (guanidinium chloride). When the optional chaotrope is employed, the same is preferably urea.

When used within a mixture to formulate an additive comprising chaotrope and active, the chaotrope and active are typically at a weight ratio from about 1:10 to about 10:1, and preferably, from about 1:5 to about 5:1, and most preferably, from about 1:2 to about 2:1, including all ratios subsumed therein. If optional chaotrope represented by formula II is used, the same typically makes up from about 0.001 to about 75%, and preferably, from about 0.1 to about 40%, and most preferably, from about 1 to about 25% by weight of chaotrope represented by the mixture of chaotropes represented as formulae I and II, including all ranges subsumed therein. In an especially preferred embodiment, however, at least about 75%, and most preferably, from about 90 to 100% by weight of the chaotrope used in this invention is represented by formula I. Chaotrope represented by formula II, nevertheless, can optionally be used to enhance active functionality.

Active suitable for used in this invention includes but is not limited to skin-lightening agents like resorcinols and niacinamide; vitamins such as vitamin C compounds including water-soluble ascorbic acid salts and esters thereof (where suitable examples include magnesium ascorboyl phosphate and the salt of the monophosphate ester of ascorbic acid), vitamin B₂, vitamin B₆, vitamin E, folic acid, biotin and vitamin E derivatives like tocopherol acetate and tocopherol palmitate; self tanning agents like dihydroxyacetone, melanin, mahakanni (eclipta alba), methyl glyoxal, erythrulose, alloxan and 2,3-dihydroxysuccindialdehyde; moisturizing agents like sorbital; deodorizing agents including ethanol and propanol; antiperspirant agents such as 1,2-hexanediol; anti-acne agents like lipoic acid, phytosterols, and salicylic acid, as well as wrinkle reducing agents such as alpha hydroxy acids, conjugated linoleic acid and petroselinic acid and active mixtures thereof. In a preferred embodiment, the active is a sunless tanning agent. In an especially preferred embodiment, the sunless tanning agent is dihydroxyacetone (DHA).

The end use composition formulated with the chaotrope and active mixture described herein typically comprises greater than 1.5 to about 45%, and preferably, greater than 1.5 to about 35%; and most preferably, from about 1.75 to about 20% by weight of the mixture of chaotrope and active, based on total weight of the end use composition and including all ranges subsumed therein. In an especially preferred embodiment, chaotrope makes up from 0.5 to 15%, and preferably, from about 1 to about 12%, and most preferably, from about 1.5 to about 6% by weight of the total weight of the end use composition, including all ranges subsumed therein.

End use compositions of the present invention can typically include a cosmetically acceptable carrier. Water is the most preferred carrier. Amounts of water may range from about 1 to about 95%, and preferably, from about 5 to about 90%, and most preferably, from about 35 to about 80% and optimally from about 40 to about 75% by weight, based on total weight of the composition and including all ranges subsumed therein. The end use compositions of this invention may be emulsions including multiple emulsions, and optionally, of the oil-in-water variety. Water-in-oil emulsions, and especially, those generally classified as water-in-oil and high internal phase emulsions are, however, preferred. Illustrative examples of the high internal phase emulsions suitable to carry the actives and chaotropes of this invention are described in commonly owned U.S. Patent Application Publication Nos. 2008/0311058 and 2009/0247445, the disclosures of which are incorporated herein by reference.

Other cosmetically acceptable carriers may include mineral oils, silicone oils, synthetic or natural esters, silicon-based polymers, fatty acids and alcohols. Amounts of these materials may range from about 0.1 to about 50%, and preferably, from about 0.1 to about 30%, and most preferably, from about 1 to about 20% by weight of the composition, including all ranges subsumed therein.

Silicone oils may be divided into the volatile and non-volatile variety. The term “volatile” as used herein refers to those materials which have a measurable vapor pressure at ambient temperature. Volatile silicone oils are preferably chosen from cyclic or linear polydimethylsiloxanes containing from about 3 to about 9, and preferably, from about 4 to about 6 silicon atoms.

Nonvolatile silicone oils useful as carrier material include polyalkyl siloxanes, polyalkylaryl siloxanes and polyether siloxane copolymers. The essentially non-volatile polyalkyl siloxanes useful herein include, for example, polydimethylsiloxanes (like dimethicone) with viscosities of from about 5 to about 100,000 centistokes at 25° C.

Among suitable esters are:

(1) Alkenyl or alkyl esters of fatty acids having 10 to 20 carbon atoms like isopropyl palmitate, isopropyl isostearate, isononyl isonanonoate, oleyl myristate, oleyl stearate, and oleyl oleate;

(2) Ether-esters such as fatty acid esters of ethoxylated fatty alcohols;

(3) Polyhydric alcohol esters such as ethylene glycol mono- and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty esters, ethoxylated glyceryl mono-stearate, 1,3-butylene glycol monostearate, 1,3-butylene glycol distearate, polyoxyethylene polyol fatty acid ester, sorbitan fatty acid esters, and polyoxy-ethylene sorbitan fatty acid esters;

(4) Wax esters such as beeswax, spermaceti, myristyl myristate, stearyl stearate; and

(5) Sterol esters, of which soya sterol and cholesterol fatty acid esters are examples thereof.

Fatty acids having from 10 to 30 carbon atoms may be included in the compositions of this invention. Illustrative of this category are pelargonic, lauric, myristic, palmitic, stearic, isostearic, hydroxystearic, oleic, linoleic, ricinoleic, arachidic, behenic and erucic acids.

Emulsifiers may be used in the end use compositions of the present invention. Total concentration of the emulsifier may range from about 0.1 to about 40%, and preferably, from about 1 to about 20%, and most preferably, from about 1 to about 10% by weight of the composition, including all ranges subsumed therein. The emulsifier may be selected from the group consisting of anionic, nonionic, cationic and amphoteric actives. Particularly preferred nonionic actives are those with a C₁₀-C₂₀ fatty alcohol or acid hydrophobe condensed with from about 2 to about 100 moles of ethylene oxide or propylene oxide per mole of hydrophobe; C₂-C₁₀ alkyl phenols condensed with from 2 to 20 moles of alkylene oxide; mono- and di-fatty acid esters of ethylene glycol; fatty acid monoglyceride; sorbitan, mono- and di-C₈-C₂₀ fatty acids; and polyoxyethylene sorbitan as well as combinations thereof. Alkyl polyglycosides and saccharide fatty amides (e.g. methyl gluconamides) are also suitable nonionic emulsifiers.

Preferred anionic emulsifiers include soap, alkyl ether sulfate and sulfonates, alkyl sulfates and sulfonates, alkylbenzene sulfonates, alkyl and dialkyl sulfosuccinates, C₈-C₂₀ acyl isethionates, C₈-C₂₀ alkyl ether phosphates, alkylethercarboxylates and combinations thereof.

Cationic emulsifiers that may be used include, for example, palmitamidopropyltrimonium chloride, distearyldimonium chloride and mixtures thereof. Useful amphoteric emulsifiers include cocoamidopropyl betaine, C₁₂-C₂₀ trialkyl betaines, sodium lauroamphoacetate, and sodium laurodiamphoacetate or a mixture thereof.

Other generally preferred emulsifiers include glyceryl stearate, glycol stearate, stearamide AMP, PEG-100 stearate, cetyl alcohol as well as emulsifying/thickening additives like hydroxyethylacrylate/sodium acryloyldimethyl taurates copolymer/squalane and mixtures thereof.

Preservatives can desirably be incorporated into the end use compositions comprising the sunless tanning agent and adjuvant of this invention to protect against the growth of potentially harmful microorganisms. Suitable traditional preservatives for compositions of this invention are alkyl esters of para-hydroxybenzoic acid. Other preservatives which have more recently come into use include hydantoin derivatives, propionate salts, and a variety of quaternary ammonium compounds. Cosmetic chemists are familiar with appropriate preservatives and routinely choose them to satisfy the preservative challenge test and to provide product stability. Particularly preferred preservatives are iodopropynyl butyl carbamate, phenoxyethanol, methyl paraben, propyl paraben, imidazolidinyl urea, sodium dehydroacetate and benzyl alcohol. The preservatives should be selected having regard for the use of the composition and possible incompatibilities between the preservatives and other ingredients in the emulsion. Preservatives are preferably employed in amounts ranging from about 0.01% to about 2% by weight of the end use composition, including all ranges subsumed therein.

Thickening agents may be included in end use compositions of the present invention. Particularly useful are the polysaccharides. Examples include starches, natural/synthetic gums and cellulosics. Representative of the starches are chemically modified starches such as sodium hydroxypropyl starch phosphate and aluminum starch octenylsuccinate. Suitable gums include xanthan, sclerotium, pectin, karaya, arabic, agar, guar, carrageenan, alginate and combinations thereof. Suitable cellulosics include hydroxypropyl cellulose, hydroxypropyl methylcellulose, ethylcellulose and sodium carboxy methylcellulose. Synthetic polymers are yet another class of effective thickening agent. This category includes crosslinked polyacrylates such as the Carbomers, polyacrylamides such as Sepigel® 305 and taurate copolymers such as Simulgel EG® and Aristoflex® AVC, the copolymers being identified by respective INCI nomenclature as Sodium Acrylate/Sodium Acryloyldimethyl Taurate and Acryloyl DimethyltaurateNinyl Pyrrolidone Copolymer. Elastomers may also be used as thickeners and these preferably include silicone elastomers.

Amounts of the thickener may range from about 0.001 to about 25%, and preferably, from about 0.5 to about 15%, and most preferably, from about 0.5 to about 10% by weight of the end use composition including all ranges subsumed therein.

Fragrances, fixatives and abrasives may optionally be included in compositions of the present invention. Each of these substances may range from about 0.05 to about 5%, preferably between 0.1 and 3% by weight.

Conventional humectants may be employed in the present invention. These are generally polyhydric alcohol-type materials. Typical polyhydric alcohols include glycerol (i.e., glycerine or glycerin), propylene glycol, dipropylene glycol, polypropylene glycol, polyethylene glycol, sorbitol, hydroxypropyl sorbitol, hexylene glycol, 1,3-butylene glycol, isoprene glycol, 1,2,6-hexanetriol, ethoxylated glycerol, propoxylated glycerol and mixtures thereof. Most preferred is glycerin, propylene glycol or a mixture thereof. The amount of humectant employed may range anywhere from 0.5 to 50%, preferably between 1 and 15% by weight of the end use composition.

Desquamation promoters may be present. Illustrative are the alpha-hydroxycarboxylic acids and beta-hydroxycarboxylic acids. The term “acid” is meant to include not only the free acid but also salts and C₁-C₃₀ alkyl or aryl esters thereof and lactones generated from removal of water to form cyclic or linear lactone structures. Representative acids are glycolic, lactic and malic acids. Salicylic acid is representative of the beta-hydroxycarboxylic acids. Amounts of these materials when present may range from about 0.01 to about 15% by weight of the end use composition.

A variety of herbal extracts may optionally be included in compositions of this invention. The extracts may either be water soluble or water-insoluble carried in a solvent which respectively is hydrophilic or hydrophobic. Water and ethanol are the preferred extract solvents. Illustrative extracts include those from green tea, yarrow, chamomile, licorice, aloe vera, grape seed, citrus unshui, willow bark, sage, thyme and rosemary.

Also, optionally suitable for use include materials like lipoic acid, retinoxytrimethylsilane (available from Clariant Corp. under the Silcare 1M-75 trademark), dehydroepiandrosterone (DHEA) and combinations thereof. Ceramides (including Ceramide 1, Ceramide 3, Ceramide 3B and Ceramide 6) as well as pseudoceramides may also be useful. Amounts of these materials may range from about 0.000001 to about 10%, preferably from about 0.0001 to about 1% by weight of the end use composition.

Conventional buffers/pH modifiers may be used. These include commonly employed additives like sodium hydroxide, hydrochloric acid, citric acid and citrate/citric acid buffers. In an especially preferred embodiment, the pH of the composition of this invention is from about 3.5 to about 7, and preferably, from about 3.75 to about 6, and most preferably, from about 4.0 to about 5.5, including all ranges subsumed therein.

Colorants, opacifiers, chelators (like tetrasodium EDTA) and abrasives may also be included in the compositions of the present invention. Each of these substances may range from about 0.05 to about 5%, preferably between 0.1 and 3% by weight of the composition.

In an especially desired embodiment, the high internal phase emulsion used in this invention comprises silicone fluids and cross-linked dimethicone-based blends, both of which are made available from Dow Corning and Shin-Etsu.

A wide variety of packaging can be employed to store and deliver the compositions. Packaging is often dependent upon the type of personal care end-use. For instance, leave-on skin lotions and creams, shampoos, conditioners and shower gels generally employ plastic containers with an opening at a dispensing end covered by a closure. Typical closures are screw-caps, non-aerosol pumps and flip-top hinged lids. Packaging for antiperspirants, deodorants and depilatories may involve a container with a roll-on ball on a dispensing end. Alternatively these types of personal care products may be delivered in a stick composition formulation in a container with propel-repel mechanism where the stick moves on a platform towards a dispensing orifice. Metallic cans pressurized by a propellant and having a spray nozzle serve as packaging for antiperspirants, shave creams and other personal care products. Toilette bars may have packaging constituted by a cellulosic or plastic wrapper or within a cardboard box or even encompassed by a shrink wrap plastic film.

When making the end use composition of the present invention, ingredients may be combined in no particular order. Typically the ingredients are combined and mixed under conditions of moderate shear and at ambient temperature with pressure being atmospheric conditions. In a most preferred embodiment, DHA and chaotrope are not added at a time when mixing and heating are desired. When applied by the consumer, typically from about 1 to 5 mg, and preferably, from about 1 to 4 mg, and most preferably, from about 1.5 to 2.5 mg per square centimeter of composition is applied to body surface (like skin) and including all ranges subsumed therein.

The following examples are provided to facilitate an understanding of the present invention. The examples are not intended to limit the scope of the claims.

### Example 1

A high internal phase emulsion (base) was made by mixing the following ingredients. Testing was conducted on vitro skin.

| Ingredients | % by weight |
|---|---|
| Water | Balance |
| Glycerin | 4 |
| Silicone emulsifier (Dow Corning DC5225C) | 3 |
| Cross-linked and alkyl modified dimethicone | 1.5 |
| blend (Shin-Etsu, KSG 340) | |
| Cross-linked dimethicone blend (Shin-Etsu, | 2 |
| KSG 210) | |
| Elastomer blend (Dow Corning, DC9045) | 5 |
| Silicone fluid (Dow Corning, DC 245) | 8 |
| Dimethicone fluid (Shin-Etsu, DMF-A6 CS) | 1.5 |
| Microspheres (Kobo Industries, CL2080) | 4 |

The base was combined with the ingredients below to make the following samples.

| 1 | 2 | 3 | 4 | 5 |
|---|---|---|---|---|
| | MgCl₂ | | | GuCl* |
| NaCl (2.5%) | (2.5%) | AlCl₃ (2.5%) | — | (2.5%) |
| DHA (2.5%) | DHA (2.5%) | DHA (2.5%) | DHA (2.5%) | DHA (2.5%) |
| Base, | Base, | Base, | Dove | Base, |
| balance | balance | balance | Energy | balance |
| | | | Glow** | |

*guanidinium chloride
**commercially available

ΔL values, using a Hunter Lab LabScan XE, were obtained for the above sample formulations. Larger ΔL values depict a darker change in color. The sample formulations were stored in a humidity controlled chamber (50%) and maintained at a temperature of about 35° C. Formulations were applied to commercially available vitro skin (2 mg/cm₂).

| | ΔL* at Time (hours) | | | | |
|---|---|---|---|---|---|
| Sample | 29 | 48 | 72 | 144 | 240 |
| 1 | 4 | 4.7 | 4.8 | 5.3 | 5.8 |
| 2 | 3.5 | 4.1 | 4.7 | 4.8 | 5.1 |
| 3 | 2.7 | 3.7 | 3.2 | 4.7 | 5.2 |
| 4 | 3.0 | 4.0 | 4.5 | 4.6 | — |
| 5 | 5.7 | 6.1 | 6.3 | 6.4 | 7.3 |

*change in L value reduction

The results indicate that compositions made with chaotrope according to the present invention, unexpectedly, resulted in superior active performance. Such a conclusion is supported by the data presented for Sample 5 where the ΔL (darkening of skin) is significant.

### Example 2

The formulations identified as Samples 4 and 5 as well as a high internal phase emulsion base with 2.5% DHA and no chaotrope were applied to pre-washed pig skin (made commercially available from Sinclair Research Center, Inc.) in lieu of vitro skin. The data below unexpectedly shows that formulations made consistent with this invention resulted in faster and greater tanning results when DHA was used as the active. Like Example 1, 2 mg/cm² of formulation was applied. Measurements for L were taken with a Smart Probe-400 color meter made available from IMS, Inc. Areas of pig skin having no formulation applied revealed essentially no color change after 23 hours. Temperature in this experiment was maintained at about 35° C., and the pig skin used was exposed to running water at about 37° C. for about 15 seconds. The washed pig skin was then wiped dry before product treatment to simulate after shower treatment.

| | ΔL* at Time (hours) | |
|---|---|---|
| Sample | 16 | 23 |
| 4 | 1.9 | 3.5 |
| 5 | 6.6 | 7.8 |
| HIPE base | 3.8 | 4.7 |

*change in L value reduction

### Example 3

The ingredients below were added to formulations having 2.5% by weight DHA and the base of Example 1 as a balance. Temperature of the formulations was maintained at about 35° C. The pig skin used was treated as described below.

| | | ΔL* | ΔL* |
|---|---|---|---|
| | | 16.5 hr | 24 hr |
| Pig Skin as supplied | No product | 1.9 | 3.7 |
| | treatment | | |
| | 2.5% | 4.9 | 6.6 |
| | guanidinium | | |
| | chloride | | |
| | 2.5% Urea | 2.7 | 4.8 |
| | 1.25% | 4.0 | 5.6 |
| | guanidinium | | |
| | chloride + | | |
| | 1.25% Urea | | |
| Pig Skin was | No product | 1.6 | 2.0 |
| exposed to running | treatment | | |
| water at 37 degrees | 1.5% GuCl | 1.8 | 2.6 |
| centigrade for 15 | 2.5% Urea | 4.4 | 6.1 |
| seconds, then wiped | 1.25% GuCl + | 3.6 | 3.8 |
| dry before product | 1.25% Urea | | |
| treatment to simulate | | | |
| after shower | | | |
| treatment. | | | |

*change in L value reduction

The results (obtained using a Smart Probe-400 Color Meter) unexpectedly show that skin darkening was enhanced when DHA was applied to pig skin (2 mg/cm²) in a formulation comprising the chaotrope of this invention.

What is claimed is:

1. A chaotrope and active additive comprising:
(a) a chaotrope represented as: 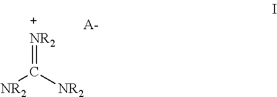
and optionally a chaotrope represented as: 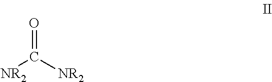
where each R is independently H, C₁₋₁₀ alkyl or aryl, and
A⁻ is a halide; and
(b) an active.

2. The chaotrope and active additive according to claim 1 wherein the chaotrope is guanidinium chloride and the active is a skin lightening, self-tanning, moisturizing, deodorizing, antiperspirant, anti-acne, or a wrinkle reducing agent or a mixture thereof.

3. The chaotrope and active additive according to claim 1 wherein the active is a self-tanning agent.

4. The chaotrope and active additive according to claim 3 wherein the self-tanning agent is dihydroxyacetone, melanin, mahakanni (eclipta alba), methyl glyoxal, erythrulose, alloxan and 2,3-dihydroxysuccindialdehyde or a mixture thereof.

5. The chaotrope and active additive according to claim 1 wherein the chaotrope and active are present at a weight ratio of about 1:10 to about 10:1 based on total weight of chaotrope and active and the chaotrope comprises guanidinium chloride.

6. The chaotrope and active additive according to claim 1 wherein the optional chaotrope is present and is urea.

7. A composition comprising the additive of claim 1.

8. The composition according to claim 7 wherein the composition is suitable for topical application to skin.

9. The composition according to claim 7 wherein the chaotrope comprises guanidinium chloride and the active is a skin lightening, self-tanning, moisturizing, deodorizing, antiperspirant, anti-acne or a wrinkle reducing agent or a mixture thereof.

10. The composition according to claim 7 wherein the active is a self-tanning agent.

11. The composition according to claim 10 wherein the self-tanning agent is dihydroxyacetone, melanin, mahakanni (eclipta alba), methyl glyoxal, erythrulose, alloxan and 2,3-dihydroxysuccindialdehyde or a mixture thereof.

12. The composition according to claim 7 wherein the chaotrope and active are present at a weight ratio of about 1:10 to about 10:1 based on total weight of chaotrope and active.

13. The composition according to claim 7 wherein the optional chaotrope is present and is urea.

14. The composition according to claim 7 wherein the composition comprises a high internal phase emulsion.

15. A method for treating skin comprising the step of topically applying to the skin the composition of claim 7.

16. The method according to claim 15 wherein the chaotrope results in an increase and faster performance of the active.
